(19) United States
(12) Patent Application Publication
(10) Pub. No.: US 2018/0311442 A1
(43) Pub. Date: Nov. 1, 2018

(54) AUTOINJECTOR

(71) Applicant: APTAR FRANCE SAS, Le Neubourg (FR)

(72) Inventors: Anthony SAUSSAYE, Louviers (FR); Franck FOUCAULT, Petit Couronne (FR)

(73) Assignee: APTAR FRANCE SAS, Le Neubourg (FR)

(21) Appl. No.: 15/772,325

(22) PCT Filed: Nov. 10, 2016
(86) PCT No.: PCT/FR2016/052924

§ 371 Date: Apr. 30, 2018

(30) Foreign Application Priority Data
Nov. 13, 2015 (FR) 1560861

(51) Int. Cl. A61M 5/31; A61M 5/315; A61M 5/20; A61M 5/32

(52) U.S. Cl. CPC A61M 5/3129; A61M 5/3157; A61M 5/2033; A61M 5/3202; A61M 2209/084; A61M 2205/581; A61M 2205/582; A61M 2005/3126; A61M 2205/583

(57) ABSTRACT

An autoinjector having a body (1) approximately cylindrical in shape and to receive a reservoir containing fluid and including a piston (P) and a needle. The body (1) has a distal portion, a middle portion, and a proximal portion, and a removable cap (20) fastened to the proximal portion. The middle portion is approximately cylindrical in shape and includes a first anti-rolling mechanism (125) forming at least one middle non-cylindrical zone in the external profile of the middle portion. The removable cap (20) is approximately cylindrical in shape and includes a second anti-rolling mechanism (21) forming at least one proximal non-cylindrical zone in the external profile of the removable cap (20). In addition or optionally, the distal portion of the body (1) is approximately cylindrical in shape and includes a third anti-rolling mechanism (115) forming at least one distal non-cylindrical zone in the external profile of the distal portion.

The present invention relates to an autoinjector.

Autoinjectors are well known in the prior art. The purpose of such devices is mainly to inject the contents of a syringe automatically into a patient's body. Various systems exist for making the penetration of the needle into the body of the patient and the injection of the fluid contained in the syringe automatic. Autoinjectors are relatively complex devices that must satisfy a certain number of constraint requirements in order to be reliable. The robustness of the device, its handling, and its ease of use for the user are also important elements. In addition, since most autoinjectors are for single use, the cost of manufacture and of assembly is also a factor that needs to be taken into account.

Numerous autoinjectors exist on the market, but they present a certain number of drawbacks.

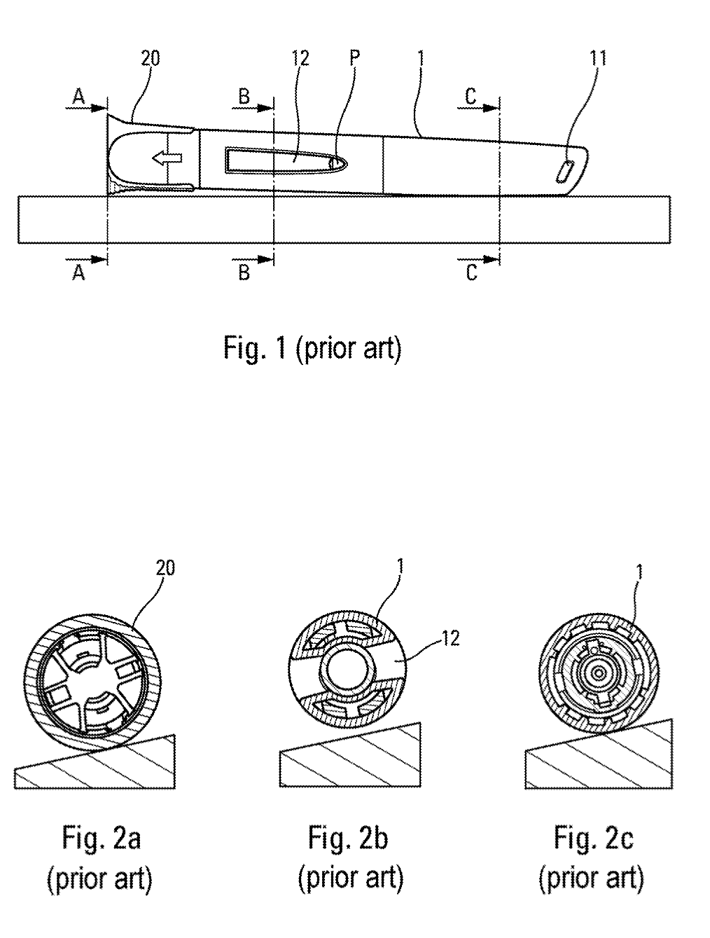

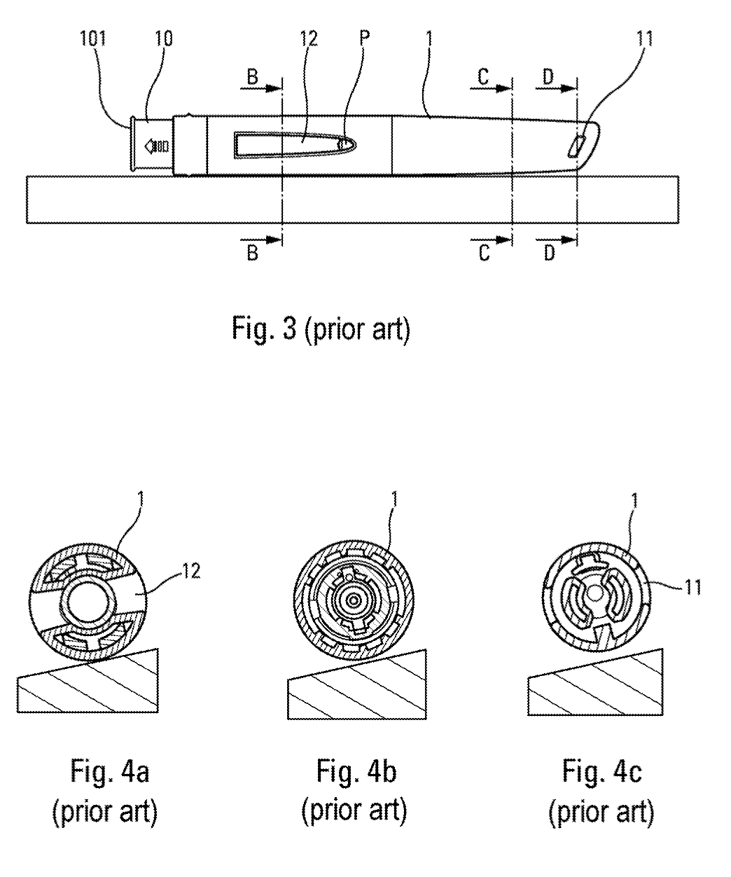

Thus, existing autoinjectors are often in the shape of an elongate cylinder. This may pose problems when the autoinjector is placed flat on a sloping surface, with the risk of the autoinjector moving by rolling about its own axis, and with a risk of the device being lost or damaged, e.g. if it falls on the floor. FIGS. 1 to 4 show this drawback. It should be observed in FIGS. 1 and 2 that while the removable cap 20 is still in place, there are two points of contact between the device and the sloping surface, namely at said cap (FIG. 2a) and at the distal portion of the body (FIG. 2c), while the middle portion is raised off said sloping surface (FIG. 2b). As a result of the circular profiles at said two points of contact, the autoinjector rolls on said sloping surface. FIGS. 3 and 4 show the situation when the removable cap 20 has been removed. It can thus be seen that only the middle portion of the body is then in contact with the sloping surface (FIG. 4a), while the proximal and distal portions (FIGS. 4b and 4c) are raised off said sloping surface. Since the profile of said middle portion is also circular, the autoinjector also rolls on said sloping surface. Thus, with or without its removable cap, the autoinjector in FIGS. 1 to 4 will not remain stationary on a sloping surface.

Another drawback with such an elongate shape may be that a user who has not been warned or who has impaired vision would risk attempting to use the autoinjector in the wrong direction, with a risk of injury by pricking by the needle and a risk of loss of dose. Thus, the FIG. 3 autoinjector, when it is used in the wrong direction, risks injuring the user with the needle,

Documents WO 2015/001280, WO 2016/050902, WO 2013/155435, WO 2011/067615, EP 2 716 318, US 2013/079718, WO 2015/036346, and FR 3 019 748 describe prior-art devices.

An object of the present invention is to provide an autoinjector that does not have the above-mentioned drawbacks.

Another object of the present invention is to provide an autoinjector that is reliable in use, that is safe and that prevents any risk of injury, and that is simple and inexpensive to manufacture, to assemble, and to use.

The present invention thus provides an autoinjector comprising a body of general shape that is approximately cylindrical and that is adapted to receive a reservoir containing fluid and including a piston and a needle, such as a pre-filled syringe, said body comprising a distal portion, a middle portion, and a proximal portion, a removable cap being fastened, before the autoinjector is used, to said proximal portion of said body, wherein:
said middle portion of said body is of general shape that is approximately cylindrical and that includes first anti-rolling means, such as at least one radially-projecting projection, forming at least one middle non-cylindrical zone in the external profile of said middle portion of said body; and
said removable cap is of general shape that is approximately cylindrical and that includes second anti-rolling means forming at least one proximal non-cylindrical zone in the external profile of said removable cap; and/or
said distal portion of said body is of general shape that is approximately cylindrical and that includes third anti-rolling means, such as at least one radially-projecting projection, forming at least one distal non-cylindrical zone in the external profile of said distal portion of said body.

Advantageously, said middle portion of said body includes at least one middle window making it possible to see the contents of said reservoir, said first anti-rolling means being formed by at least one radially-projecting edge of said at least one middle window.

Advantageously, said at least one radially-projecting edge surrounds said at least one middle window completely.

In a variant, said at least one radially-projecting edge surrounds said at least one middle window in part only.

Advantageously, said middle portion of said body includes two diametrically-opposite middle windows, each including said first anti-rolling means.

Advantageously, said removable cap includes a radially-outer portion of circular shape, said second anti-rolling means being formed by at least one flat that is formed in said radially-outer portion of circular shape.

Advantageously, said second anti-rolling means are formed by two diametrically-opposite flats.

Advantageously, said distal portion of said body includes at least one distal window making it possible to display a visual indicator of an end-of-injection indicator device, said third anti-rolling means being formed by at least one radially-projecting edge of said at least one distal window.

Advantageously, said at least one radially-projecting edge surrounds said at least one distal window completely.

In a variant, said at least one radially-projecting edge surrounds said at least one distal window in part only.

Advantageously, said distal portion of said body includes two diametrically-opposite distal windows, each including said third anti-rolling means.

Advantageously, said autoinjector includes an actuator sleeve that is movable relative to said body between projecting positions in which said actuator sleeve projects out from said body at least in part, and an actuated position in which said actuator sleeve is moved axially into said body, said actuator sleeve being in a first projecting position before actuation of the autoinjector, and in a second projecting position after actuation of the autoinjector, said actuator sleeve being urged towards said projecting positions by a spring.

Advantageously, said actuator sleeve includes a contact end for coming into contact with the injection zone.

These characteristics and advantages and others of the present invention appear more clearly from the following detailed description, given by way of non-limiting examples, and with reference to the accompanying drawings, and in which:

FIG. 1 is a diagrammatic side view of a prior-art autoinjector, before removal of the protective cap;

FIG. 2a is a diagrammatic section view on line A-A of FIG. 1, with the autoinjector placed on a sloping surface;

FIG. 2b is a diagrammatic section view on line B-B of FIG. 1, with the autoinjector placed on a sloping surface;

FIG. 2c is a diagrammatic section view on line C-C of FIG. 1, with the autoinjector placed on a sloping surface;

FIG. 3 is a diagrammatic side view of the FIG. 1 autoinjector, after removal of the protective cap;

FIG. 4a is a diagrammatic section view on line B-B of FIG. 3, with the autoinjector placed on a sloping surface;

FIG. 4b is a diagrammatic section view on line C-C of FIG. 3, with the autoinjector placed on a sloping surface;

FIG. 4c is a diagrammatic section view on line D-D of FIG. 3, with the autoinjector placed on a sloping surface;

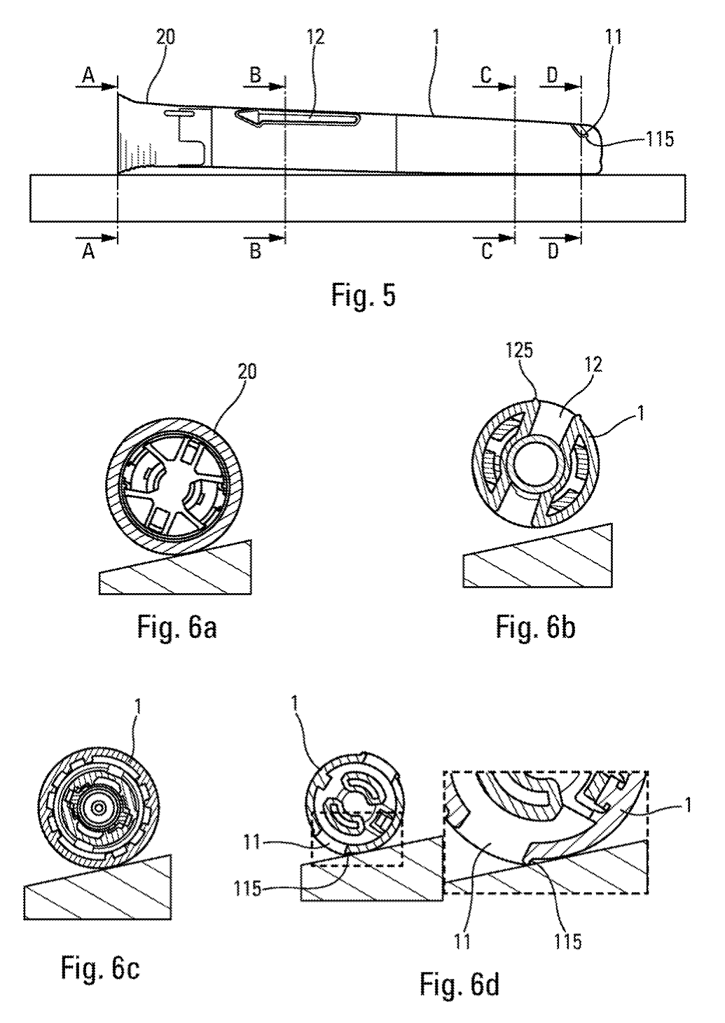

FIG. 5 is a diagrammatic side view of an autoinjector constituting an advantageous embodiment of the present invention, before removal of the protective cap;

FIG. 6a is a diagrammatic section view on line A-A of FIG. 5, with the autoinjector placed on a sloping surface;

FIG. 6b is a diagrammatic section view on line B-B of FIG. 5, with the autoinjector placed on a sloping surface;

FIG. 6c is a diagrammatic section view on line C-C of FIG. 5, with the autoinjector placed on a sloping surface;

FIG. 6d is a diagrammatic section view on line D-D of FIG. 5, with the autoinjector placed on a sloping surface;

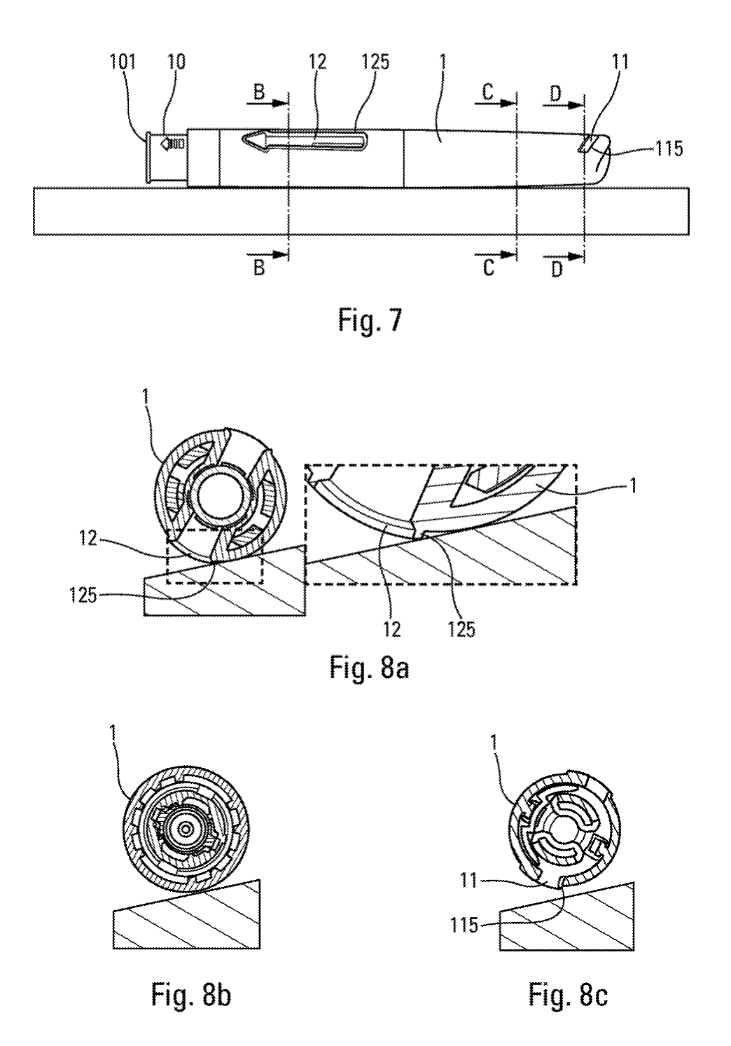

FIG. 7 is a diagrammatic side view of the FIG. 5 autoinjector, after removal of the protective cap;

FIG. 8a is a diagrammatic section view on line B-B of FIG. 7, with the autoinjector placed on a sloping surface;

FIG. 8b is a diagrammatic section view on line C-C of FIG. 7, with the autoinjector placed on a sloping surface;

FIG. 8c is a diagrammatic section view on line D-D of FIG. 7, with the autoinjector placed on a sloping surface;

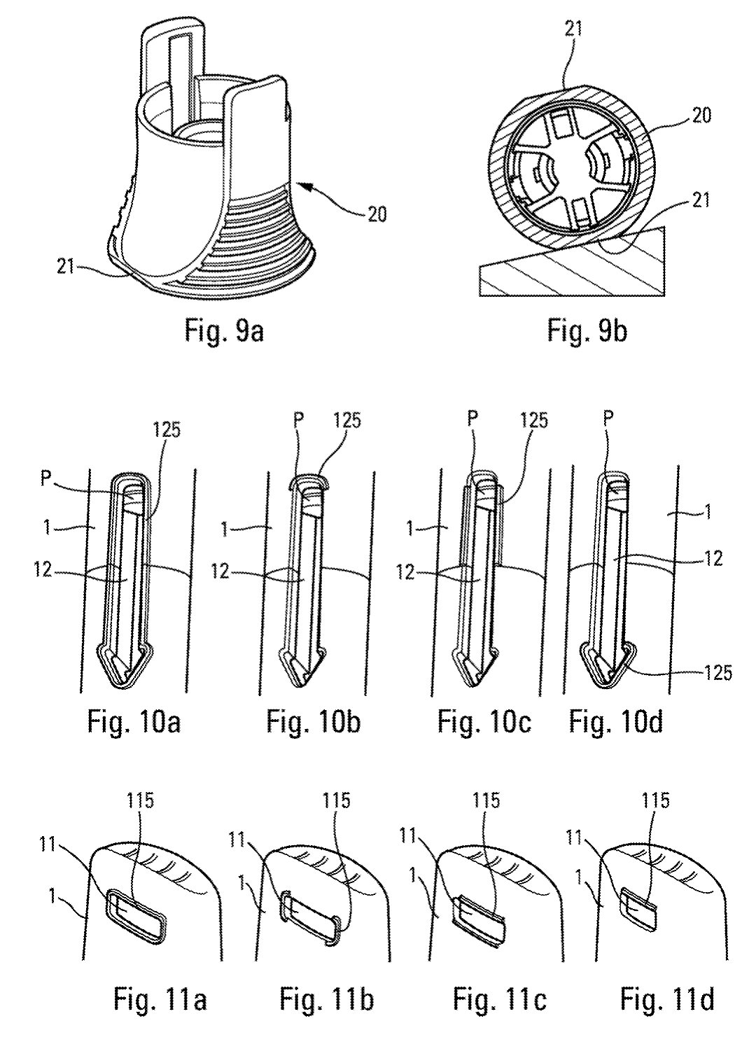

FIG. 9a is a perspective view of a removable cap in a variant embodiment;

FIG. 9b is a view similar to the view in FIG. 6a, with the FIG. 9a removable cap;

FIGS. 10a to 10d are diagrammatic side views of four variant embodiments;

FIGS. 11a to 11d are diagrammatic side views of four other variant embodiments; and

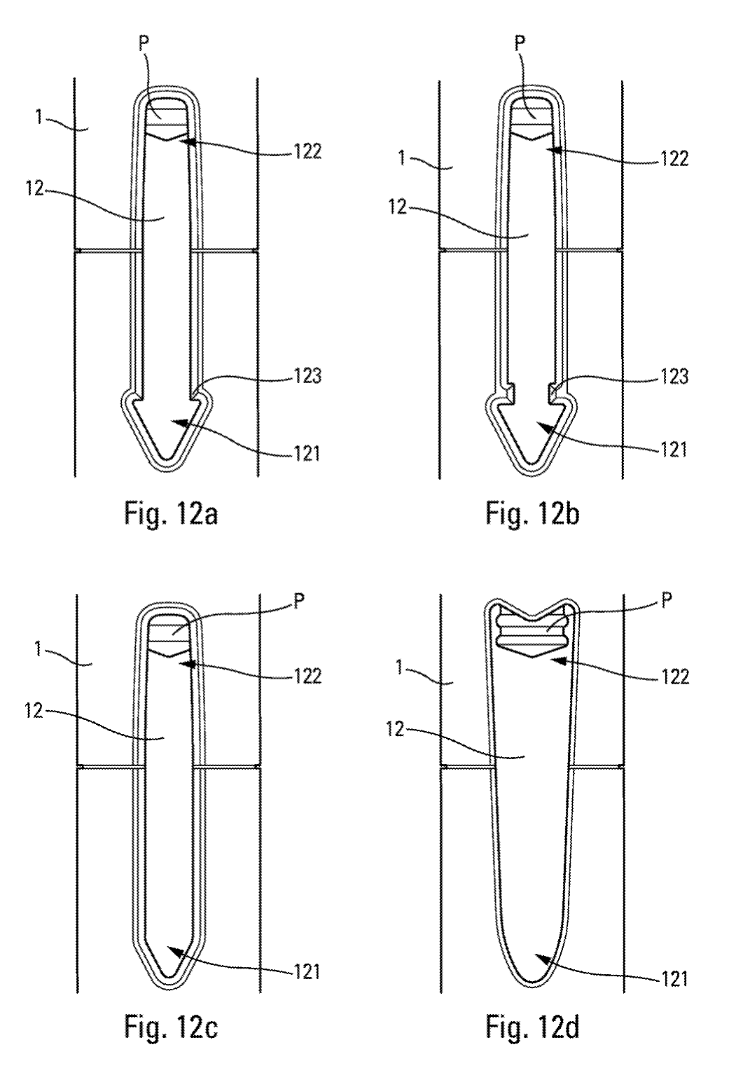

FIGS. 12a to 12d are diagrammatic side views of four other variant embodiments.

In the following description, the terms “proximal” and “distal” are relative to the needle of the autoinjector. The terms “axial” and “radial” refer to the longitudinal central axis of the autoinjector. The terms “top” and “bottom” refer to the orientation in FIGS. 11a to 11d.

The autoinjector is described below with reference to advantageous embodiments. It should nevertheless be observed that autoinjectors, which are complex appliances, comprise a plurality of modules for performing a plurality of functions. The various modules may be used separately and independently of one another, without necessarily being combined with the other modules, and in particular they could be used in autoinjectors of shape that is different from the shape shown in the drawings. Furthermore, it should be observed that the drawings are diagrammatic views, which do not necessarily represent the exact shape of the components of an autoinjector, and they are not necessarily to scale, in particular for purposes of clarity. In addition, the drawings do not represent all of the component elements of an autoinjector, but only the elements necessary for operation of the present invention. Thus, the internal structures of the autoinjector, which do not affect the present invention, are not shown in the drawings for the sake of clarity, and they are described only briefly below. In addition, various additional and/or complementary elements and modules could be associated with the autoinjector shown in the figures.

The autoinjector shown in the figures comprises a body 1 of general shape that is approximately cylindrical and in which an actuator sleeve 10 slides axially, the actuator sleeve 10 having a bottom end 101 that is for coming into contact with the body of the patient around the injection zone. Advantageously, the autoinjector may include a lower body, an intermediate body, and an upper body that are assembled together so as to form the body 1 of the autoinjector. Below, the term “body” and the numerical reference “1” are used to designate said unitary body formed by assembling said lower body with said intermediate body and said upper body. It should be observed that the body 1 could be formed of any number of body portions, e.g. two.

The body 1 includes a proximal portion, at the needle end, on which there is assembled a removable cap 20 that the user must remove before actuation. The body 1 also includes a distal portion that is axially remote from said needle and that is advantageously provided with at least one distal window 11, and a middle portion that is advantageously provided with at least one middle window 12. Said proximal, middle, and distal portions of the body 1 are of general shape that is approximately cylindrical. Likewise, the removable cap 20 has a shape that is approximately circularly cylindrical. Thus, with or without said removable cap, the autoinjector has an elongate shape that is approximately cylindrical and that, in the absence of the present invention, would be likely to roll when placed on a sloping plane.

A reservoir (not shown) is inserted into said autoinjector. The reservoir contains fluid and includes a piston P and a needle A. The piston P is adapted to move in said reservoir so as to inject the fluid through said needle. The present description is made with reference to a syringe that may be of any type. More generally, it is understood that the term “syringe” in the present description encompasses any type of reservoir associated with a needle. Preferably, the reservoir is a pre-filled syringe. Said reservoir can be seen through said at least one middle window 12, as shown in FIGS. 1, 3, 10, and 12. Advantageously, there are two diametrically-opposite middle windows 12 on the middle portion of the body 1. Said at least one middle window 12 makes it possible to see 100% of the liquid contained in the reservoir before use. It also makes it possible to see the piston at the end of injection. It may also show the piston before use.

The autoinjector also includes an automatic injection system, in particular comprising a piston rod (not shown) that is adapted to co-operate with the piston P so as to move it in the reservoir so as to dispense the fluid through the needle. Conventionally, the piston rod is urged by an injection spring (not shown) towards its dispensing position and, before actuation, it is held in its rest position by an appropriate injection lock. Examples of advantageous injection locks are described in particular in documents WO 2013/175148 and WO 2015/155484.

The autoinjector may also include a visual, audible, and/or tactile indicator device for indicating to the user, in particular by an audible sound, by vibration, and/or by visual and/or tactile indication, that the autoinjector may be removed from the injection site. In particular, the indicator device may include one or more indicator elements that give both a visual indication, by a suitable display in said at least one distal window 11 of the body 1, and also an audible and/or tactile indication. Advantageously, there are two diametrically-opposite distal windows 11 on the distal portion of the body 1.

The actuator sleeve 10 is urged towards its projecting positions by a spring (not shown) that may be of any type. The spring co-operates both with said actuator sleeve 10 and also with a portion that is secured to the body 1. Before actuation, the actuator sleeve 10 is in a first projecting position in which it surrounds the needle, as shown in FIG. 7. During actuation, the actuator sleeve 10 slides inside the body 1 towards an actuated position, so as to expose the needle and enable pricking, and then injection of the fluid. After injection, when the user removes the autoinjector from the injection site, the actuator sleeve 10 returns into an end-of-use second projecting position in which it is once again arranged around the needle, so as to avoid any risk of injury with said needle. It should be observed that the first and second projecting positions of the actuator sleeve 10 may be positions that are different or that are identical.

Before the autoinjector is used, the needle of the syringe is advantageously protected by a guard, typically made out of elastomer, into which the end of the needle is poked. In this configuration, removing said removable cap 20 advantageously causes said guard to be removed from the needle.

When the user wishes to use the autoinjector, the user takes hold of the device, e.g. at the body 1, and presses the actuator sleeve 10 against the part of the body where the injection is to be performed. The pressure exerted by the user on the actuator sleeve 10 causes said actuator sleeve to slide inside the body 1, thereby uncovering the needle and thus pricking the user as a result of the pressure exerted by the user on the autoinjector.

When the actuator sleeve 10 reaches its actuated position, which is its end position inside the body 1, it causes the injection stage to be triggered, during which the piston rod slides inside the syringe, pushing its piston P under the effect of the injection spring. The fluid is thus dispensed.

At the end of injection, the indicator device is actuated, possibly after a predetermined delay, so as to indicate to the user that the autoinjector may be removed from the injection site. The actuator sleeve 10 thus once again moves out from the body 1 towards the second projecting position, under the effect of its spring, with said actuator sleeve 10 being locked, and this guarantees absolute safety for the user and avoids any risk of injury with the needle after the device has been used.

In an advantageous aspect, the autoinjector includes at least two, advantageously three, anti-rolling means that prevent said autoinjector from rolling on a sloping surface, with or without removable cap 20.

Thus, said middle portion of said body 1, that is of general shape that is approximately cylindrical, includes first anti-rolling means 125, forming at least one middle non-cylindrical zone in the external profile of said middle portion of said body 1. The first anti-rolling means 125 prevent any rolling after removal of the removable cap 20, as shown in FIGS. 7 and 8. It should be observed in FIG. 7 that, in the absence of the removable cap 20, the proximal and distal portions of the body 1 are not in contact with the support surface. This is shown in particular in FIGS. 8b and 8c. It is thus only the middle portion of the body 1 that is in contact with the surface, and when the surface slopes, the first anti-rolling means 125 prevent the autoinjector from rolling, as can be seen in FIG. 8a, having a right-hand portion that shows a detail of the left-hand portion on a larger scale.

Preferably, said first anti-rolling means 125 are formed by at least one projection that projects radially out from the approximately-cylindrical external profile of said middle portion of said body 1.

Advantageously, said first anti-rolling means 125 are formed by at least one radially-projecting edge of said at least one middle window 12. FIGS. 10a to 10d show four variant embodiments. In FIG. 10a, said radially-projecting edge surrounds said middle window 12 completely. In FIG. 10b, said radially-projecting edge surrounds only the distal portion of said middle window 12, whereas in FIGS. 10c and 10d, it is the middle and proximal portions respectively of said middle window 12 that include respective radially-projecting edges. Naturally, other variants are possible. Advantageously, two diametrically-opposite middle windows 12 are provided, each including first anti-rolling means 125.

As can be seen in FIG. 8a, said radially-projecting edge forms a radial shoulder that prevents any turning of the autoinjector.

As can be seen in FIGS. 5 and 6b, said middle portion of the body 1, and in particular said middle window 12, is not in contact with the support surface when the removable cap 20 is in place. The first anti-rolling means 125 are thus not effective in this circumstance. It is thus necessary for the autoinjector to include other anti-rolling means that act when the removable cap 20 is assembled on the autoinjector.

In a first variant, said removable cap 20 includes second anti-rolling means 21 forming at least one proximal non-cylindrical zone in the generally approximately-cylindrical external profile of said removable cap 20. Advantageously, said removable cap 20 includes a radially-outer portion of circular shape, said second anti-rolling means 21 being formed by at least one flat that is formed in said radially-outer portion of circular shape. This is shown in FIGS. 9a and 9b. Preferably, two diametrically-opposite flats are provided on said removable cap 20. Naturally, other variants are possible, e.g. radial projections in place of flats.

In a second variant, said distal portion of general shape that is approximately cylindrical of said body 1 includes third anti-rolling means 115, forming at least one distal non-cylindrical zone in the external profile of said distal portion of said body 1.

Preferably, said third anti-rolling means 115 are formed by at least one projection that projects radially out from the approximately-cylindrical external profile of said distal portion of said body 1.

Advantageously, said third anti-rolling means 115 are formed by at least one radially-projecting edge of said at least one distal window 11. FIGS. 11a to 11d show four variant embodiments. In FIG. 11a, said radially-projecting edge surrounds said distal window 11 completely. In Figure lib, said radially-projecting edges are formed only on the lateral sides of said distal window 11, whereas in FIG. 11c, it is the top and bottom sides of said distal window 11 that each includes a radially-projecting edge. Finally, FIG. 11d shows a radially-projecting edge formed only on the top side of the distal window 11. Naturally, other variants are possible. Advantageously, two diametrically-opposite distal windows 11 are provided, each including third anti-rolling means 115.

As can be seen in FIG. 6d, said radially-projecting edge forms a radial shoulder that prevents any turning of the autoinjector.

Naturally, it is possible to combine the two variants mentioned above.

In another advantageous aspect, said at least one middle window 12 has an external shape in the shape of an arrow pointing towards the proximal portion of said body 1. The purpose of this configuration is to avoid using the autoinjector in the wrong direction. The arrow shape clearly indicates to the user in which direction it is necessary to actuate the device.

Advantageously, the arrow shape can also be detected easily by people with impaired vision, in particular when said middle window 12 includes a radially-projecting edge, as described above.

FIGS. 12a to 12d show four variant embodiments.

In the variants in FIGS. 12a and 12b, the proximal portion 121 of the middle window 12 includes a triangular end portion, forming the point of the arrow, connected to the remainder of the window via a shoulder 123. FIG. 12b includes a shoulder 123 that is larger.

FIG. 12c also includes a triangular proximal end portion 121, but without a shoulder 123.

Finally, FIG. 12d includes a proximal window portion 121 that is more rounded, but that still points towards the proximal portion of the body 1. In this embodiment, the distal portion 122 of the window 12 includes a profile in the shape of a point that also points towards the proximal portion of the body 1, and this reinforces the visual impression of a middle window 12 that points towards the needle. Naturally, the distal portion 122 of FIG. 12d could be associated with the other variants in FIGS. 12a to 12c.

Naturally, other variants are possible.

The present invention applies to devices used in particular for treatment of auto-immune diseases, e.g. of the rheumatoid arthritis, multiple scleroses, Crohn's disease type, for treatment of cancer, for antiviral treatments, e.g. of the hepatitis type, for treatment of diabetes, for treatment of anemia, or for treatment of allergy attacks, e.g. in the event of anaphylactic shock.

Although the present invention is described above with reference to advantageous embodiments, naturally various modifications can be applied thereto by the person skilled in the art, without going beyond the ambit of the present invention, as defined by the accompanying claims.

1. An autoinjector comprising a body (1) in the shape of an elongate cylinder that is adapted to receive a reservoir containing fluid and including a piston (P) and a needle, said body (1) comprising a distal portion of circular shape, a middle portion, and a proximal portion, a removable cap (20) being fastened, before the autoinjector is used, to said proximal portion of said body (1), said middle portion of said body (1) of circular shape including first anti-rolling means (125) formed by at least one radially-projecting projection in the circular external profile of said middle portion of said body (1), said at least one radially-projecting projection forming at least one middle non-cylindrical zone in the external profile of said middle portion of said body, wherein said removable cap (20) includes a radially-outer portion of circular shape and second anti-rolling means (21) forming at least one proximal non-cylindrical zone in the circular external profile of said removable cap (20), and in that said distal portion of said body (1) includes third anti-rolling means (115) forming at least one distal non-cylindrical zone in the circular external profile of said distal portion of said body (1).

2. An autoinjector according to claim 1, wherein said middle portion of said body (1) includes at least one middle window (12) making it possible to see the contents of said reservoir, said first anti-rolling means (125) being formed by at least one radially-projecting edge of said at least one middle window (12).

3. An autoinjector according to claim 2, wherein said at least one radially-projecting edge surrounds said at least one middle window (12) completely.

4. An autoinjector according to claim 2, wherein said at least one radially-projecting edge surrounds said at least one middle window (12) in part only.

5. An autoinjector according to claim 1, wherein said second anti-rolling means (21) are formed by at least one flat that is formed in said radially-outer portion of circular shape.

6. An autoinjector according to claim 1, wherein said distal portion of said body (1) includes at one distal window (11) making it possible to display a visual indicator of an end-of-injection indicator device, said third anti-rolling means (115) being formed by at least one radially-projecting edge of said at least one distal window (11).

7. An autoinjector according to claim 6, wherein said at least one radially-projecting edge surrounds said at least one distal window (11) completely.

8. An autoinjector according to claim 6, wherein said at least one radially-projecting edge surrounds said at least one distal window (11) in part only.

9. An autoinjector according to claim 1, wherein said autoinjector includes an actuator sleeve (10) that is movable relative to said body (1) between projecting positions in which said actuator sleeve (10) projects out from said body (1) at least in part, and an actuated position in which said actuator sleeve (10) is moved axially into said body (1), said actuator sleeve (10) being in a first projecting position before actuation of the autoinjector, and in a second projecting position after actuation of the autoinjector, said actuator sleeve (10) being urged towards said projecting positions by a spring.

10. An autoinjector according to claim 9, wherein said actuator sleeve (10) includes a contact end (101) for coming into contact with the injection zone.

11. The autoinjector according to claim 1, wherein the piston and needle art part of a pre-filled syringe.
